(19) United States
(12) Patent Application Publication
(10) Pub. No.: US 2011/0257125 A1
(43) Pub. Date: Oct. 20, 2011

(54) Mucomimetic compositions and uses therefore

(76) Inventor: Rolf Schaefer, Arisdorf (CH)

(21) Appl. No.: 13/067,762

(22) Filed: Jun. 24, 2011

Related U.S. Application Data
(63) Continuation-in-part of application No. 12/285,350, filed on Oct. 2, 2008.

(51) Int. Cl. A61K 31/728; A61P 27/02

(52) U.S. Cl. 514/54

(57) ABSTRACT

The present invention relates to mucomimetic and ophthalmic solutions comprising a cationic multimeric antimicrobial agent such as polyaminopropyl biguanide and a magnesium, calcium or magnesium/calcium complex of an anionic polymer such as hyaluronate, alginate, carboxymethyl cellulose, chondroitin sulfate or mixtures thereof. In specific embodiments, the solutions include additional components such as a surfactant, a viscosity-modifying agent, a tonicity agent and a buffer. The solutions are biocompatible with and are highly comfortable when administered to mucous membranes, including those of the eye, as well as are effective disinfectants.

### CROSS-REFERENCE TO RELATED APPLICATIONS

This application is a continuation-in-part of U.S. application Ser. No. 12/285,350, filed on Oct. 2, 2008.

### FIELD OF THE INVENTION

The invention relates to mucomimetic compositions wherein a magnesium, calcium or magnesium/calcium complex of an anionic polymeric substance, especially hyaluronic acid, carboxymethylcellulose, alginate and/or chondroitin sulfate, is combined with a cationic polymeric antimicrobial agent such as polyaminopropyl biguanide, wherein said compositions provide comfort and biocompatibility with mucous membranes without substantially affecting antimicrobial efficacy of the cationic polymeric antimicrobial agent.

### BACKGROUND OF THE INVENTION

Anionic polymers such as hyaluronic acid and carboxymethylcellulose are known for their moisturizing and lubricating properties. Such properties generally result in a reduction in irritation to mucous membranes, including those of the eye. Cationic antimicrobial agents have also been noted as beneficial to include in ophthalmic solutions. However, these two types of components were not understood as being compatible within one solution.

Cationic antimicrobial disinfecting agents were found to be compromised in their efficacy in the presence of certain anionic entities. For example, U.S. Pat. No. 5,858,346 teaches that polyaminopropyl biguanide and other non-oxidative disinfectants (typically cationic entities) are neutralized in their ability to damage cell walls, including cell walls of microorganisms, when combined with carboxymethyl cellulose and/or various other negatively charged entities. Although the neutralization of the polyaminopropyl biguanide or other non-oxidative disinfectants alleviates irritation of mucous membranes (i.e., the eye), the neutralization also results in loss of antimicrobial efficacy.

Similarly, U.S. Pat. No. 5,559,104, to Romeo et al., teaches the use of cetylpyridinium chloride as an ion-pairing agent for the precipitation and purification of hyaluronic acid in a proposed manufacturing process for the hyaluronic acid biopolymer. Thus, that cetylpyridinium chloride would retain its antimicrobial activity in the presence of hyaluronic acid or carboxymethyl cellulose is counterintuitive. However, Powell and Huth were recently able prepare ophthalmic solutions comprising an anionic polymer and a cationic monomeric or dimeric antimicrobial agent, in which activity of the antimicrobial agent was retained. U.S. patent application Ser. No. 11/271,448. It is noted that cationic polymeric antimicrobial agents were discussed. However, no composition containing such antimicrobial agent was proposed, suggesting that the authors were unable to or considered it not possible to prepare such a composition without concomitant loss of antimicrobial activity.

### SUMMARY OF THE INVENTION

The present invention relates to mucomimetic solutions comprising a cationic multimeric antimicrobial agent and a magnesium, calcium or magnesium/calcium complex of an anionic polymer. In one set of embodiment, the mucomimetic solution is an ophthalmic solution comprising a cationic multimeric antimicrobial agent and a magnesium, calcium or magnesium/calcium complex of an anionic polymer but not myristamidopropyl dimethylamine or a nutritive base comprising a multiplicity of amino acids and vitamins, wherein the cationic multimeric antimicrobial agent is present in an amount ranging from about 0.0001 to about 0.0005% w/v (weight/volume) and the magnesium, calcium or magnesium/calcium complex of an anionic polymer in an amount ranging from about 0.01 to about 0.25% w/v. In preferred embodiments, the cationic multimeric antimicrobial agent is polyaminopropyl biguanide or an acceptable salt thereof or the anionic polymer is selected from the group consisting of hyaluronate, alginate, carboxymethyl cellulose, chondroitin sulfate and mixtures thereof. More preferably, the anionic polymer is hyaluronate. In more preferred embodiments, the anionic polymer has a molecular weight of from about 70,000 to about 4 million Daltons. The ophthalmic solutions of the invention can include some (one or more) or, preferably, all of the following additional components. A first such additional component is a viscosity-modifying agent. that can be included in an amount ranging from about 0.01 to about 0.2% w/v and can be selected from the group consisting of cellulose polymers including hydroxypropylmethyl cellulose, hydroxyethyl cellulose, ethylhydroxyethyl cellulose, hydroxypropyl cellulose, methyl cellulose, glycerol, carbomers, polyvinyl alcohol, polyvinyl pyrrolidone, carrageenans, guar, karaya, agarose, locust bean, tragacanth and xanthan gums. Preferred viscosity-modifying agents are hydroxypropylmethyl cellulose and hydroxyethyl cellulose. Another additional component is a surfactant that is included in an amount ranging from about 0.01 to about 1% w/v. The surfactant can be selected from the group consisting of polyethylene oxide, polypropylene oxide, polyoxamers, polyoxamines, polysorbate 20 and polysorbate 80. A preferred surfactant is polysorbate 20. A further additional component is a buffer, added in an amount ranging from about 0.01 to about 0.25% w/v. The buffer can be selected from the group of buffers consisting of borate, citrate, acetate, histidine, tris, bis-tris and mixtures thereof, with borate buffers being most preferred. Another additional component is a tonicity agent that is included in an amount ranging from about 0.001 to about 1% w/v. The tonicity agent can be selected from the group consisting of sodium chloride, potassium chloride, dextrose, glycerol, propylene glycol, mannitol, sorbitol and mixtures thereof. Preferred are sodium chloride or mixtures of sodium chloride and potassium chloride. The pH of the ophthalmic solutions preferably is adjusted to range from 6.5 to 8.5

In a more specific preferred embodiment, an ophthalmic composition of the invention either comprises or is an aqueous solution of (a) polyaminopropyl biguanide or an acceptable salt thereof in an amount ranging from about 0.0001 to about 0.0005% w/v; (b) a magnesium, calcium or magnesium/calcium complex of an anionic polymer in an amount ranging from about 0.01 to about 0.25% w/v, whereby the latter complex has a molecular weight of between about 70,000 and about 4 million Dalton; (c) a viscosity-modifying agent in an amount ranging from about 0.01 to about 0.2% w/v; (d) a surfactant in an amount ranging from about 0.01 to about 1% w/v; (e) a tonicity agent to adjust tonicity to between 150 and 450 mOsm/kg ranging from about 0.001 to about 1% w/v; and (f) a buffer in an amount ranging from about 0.01 to about 0.25% w/v; whereby the pH of the aqueous solution is adjusted to be from about 6.5 to about 8.5. In even more preferred specific embodiments of the latter ophthalmic composition, the anionic polymer is hyaluronate. The viscosity-modifying agent can be selected from the group consisting of cellulose polymers including hydroxypropylmethyl cellulose, hydroxyethyl cellulose, ethylhydroxyethyl cellulose, hydroxypropyl cellulose, methyl cellulose, glycerol, carbomers, polyvinyl alcohol, polyvinyl pyrrolidone, carrageenans, guar, karaya, agarose, locust bean, tragacanth and xanthan gums. Most preferably, it is either hydroxypropylmethyl cellulose or hydroxyethyl cellulose. The surfactant can be selected from the group consisting of polyethylene oxide, polypropylene oxide, polyoxamers, polyoxamines, polysorbate 20 and polysorbate 80. Most preferably, it is polysorbate 20. The buffer can be selected from the group of buffers consisting of borate, citrate, acetate, histidine, tris, bis-tris and mixtures thereof. Most preferably, it is a borate buffer. The tonicity agent can be sodium chloride, potassium chloride, dextrose, glycerol, propylene glycol, mannitol, sorbitol and mixtures thereof. Most preferably, it is sodium chloride or a mixture of sodium chloride and potassium chloride.

Another set of embodiments encompasses mucomimetic compositions that are identical to the above-described ophthalmic solutions, except that the cationic multimeric antimicrobial agent is present in an amount ranging from about 0.001 to about 0.01% w/v. These solutions are used in non-ophthalmological applications.

### DETAILED DESCRIPTION OF THE INVENTION

Novel mucomimetic solutions comprising a complex of a negatively charged polymeric substance and a cationic multimeric antimicrobial agent are described, as well as their methods of use and preparation. Solutions according to the present invention may be used as ophthalmic solutions for effective contact lens disinfection compositions, lens cleansing solution compositions, lens packaging solution compositions, eye drops such as rewetters and tears or as vehicles for topical delivery of active substances to the eye. The compositions provide additional comfort to the eye. Furthermore, they are biocompatible with lenses and, unlike prior art compositions, do not cause a discoloring of soft contact lenses. The efficacy of the antimicrobial agent is retained in the compositions of the invention. The compositions of the invention may also be used in wipes, solutions and lubricating products that are brought in contact with other mucous membranes of a subject. They can be used for moisturing, lubricating, cleaning or disinfection purposes and may also serve as vehicles for delivering an active substance into or through the latter mucous membranes.

It has been discovered that certain anionic polymeric substances in the form of supramolecular complexes with magnesium and/or calcium ions, are highly comfortable to the eye and do not inactivate cationic multimeric antimicrobial agents such as polyaminopropyl biguanide. The concentration (w/v) of the cationic multimeric antimicrobial agent in ophthalmic solutions of the invention is preferably at least about ten fold lower and more preferably at least about hundred fold lower than that of the anionic polymer. In general purpose formulations that are not for ophthalmological use, the concentration ratio (w/v) of magnesium or calcium complex of anionic polymer to cationic antimicrobial agent may typically be lower, i.e., between 10:1 and 1:1. The anionic polymeric substances used in the solutions of the invention include hyaluronate, alginate, carboxymethyl cellulose, chondroitin sulfate and mixtures thereof.

A magnesium and/or calcium complex of an anionic polymeric substance is typically prepared by co-dissolving (in any order) in an aqueous solution an anionic polymer such as, e.g., hyaluronic acid or sodium hyaluronate, and a magnesium or calcium salt, e.g., magnesium chloride or calcium chloride. The amount of magnesium chloride or calcium chloride added is sufficient to neutralize all carboxy groups in the hyaluronate (or negatively charged groups in another anionic polymer). The aqueous solution in which anionic polymeric substance and metal salt(s) are dissolved may already contain a cationic antimicrobial agent such as polyaminopropyl biguanide. Alternatively, the polyaminopropyl biguanide is added along with or subsequent to the addition of anionic polymer and metal salt. Neutralization of polyaminopropyl biguanide resulting in loss of antimicrobial activity is prevented because magnesium and/or calcium ions are better electrophiles than polyaminopropyl biguanide. Hence, a magnesium and/or calcium complex of anionic polymer is formed rather then a complex containing polyaminopropyl biguanide and anionic polymer.

The compositions of the invention are compatible with typical additives to mucomimetic and ophthalmic compositions, such as typical buffer systems, surfactants, tonicity agents and viscosity-modifying agents.

In one embodiment, a composition of the invention comprises a magnesium, calcium or magnesium/calcium complex of at least one anionic polymeric substance, such as hyaluronate, alginate, carboxymethyl cellulose, chondroitin sulfate or mixtures thereof, and at least one cationic multimeric antimicrobial agent such as polyaminopropyl biguanide or an acceptable salt thereof. In this and any other embodiment, a composition of the invention does not contain myristamidopropyl dimethylamine or myristamidopropyl dimethylamine dimethicone copolyol phosphate. Addition of such antimicrobial agent is not required in the case of the compositions of the inventions that comprise a cationic multimeric antimicrobial agent such as polyaminopropyl biguanide in a non-neutralized form. Furthermore, the compositions do not include a nutritive base comprising a multiplicity of amino acids and vitamins such as described by Thorel and Gatto in U.S. Patent Publication No. 2006/0103807.

In a preferred embodiment, the composition is comprised of specific concentration ranges of said cationic multimeric antimicrobial agent, preferably polyaminopropyl biguanide or an acceptable salt thereof, and said complex of a water-soluble anionic polymeric substance. A typical concentration range for the cationic multimeric antimicrobial agent is from about 0.001% to about 0.01% w/v for a general purpose solution and from about 0.0001 to about 0.0005% w/v for an ophthalmic solution. The range for the complexed anionic polymeric substance is from about 0.01% to about 0.25% w/v. Preferably, the anionic polymeric substance has a molecular weight of from about 70,000 to about 4 million Daltons.

In a more preferred embodiment, a mucomimetic or ophthalmic composition of the invention is comprised of polyaminopropyl biguanide or an acceptable salt thereof such as a borate salt, and a magnesium, calcium or magnesium/calcium (any ratio) complex of hyaluronate.

The compositions of the present invention typically include a buffer component. The present compositions may have a pH that is compatible with the intended use, and is often between about 5 and 9. A variety of conventional buffers may be employed, such as borate, citrate, acetate, histidine, tris, bis-tris and the like and mixtures thereof. Borate buffers include boric acid and its salts, such as sodium or potassium borate. Potassium tetraborate or potassium metaborate, which produce boric acid or a salt of boric acid in solution, may also be employed. Hydrated salts such as sodium borate decahydrate can also be used. Additionally, organic counter-ions for the above buffers may also be employed. The concentration of buffer generally ranges from about 0.01 to 0.25% w/v.

The type and amount of buffer are selected so that the composition meets the functional performance criteria of the composition, such as physicochemical attributes and shelf life stability, antimicrobial efficacy, buffer capacity and the like factors. The buffer is also selected to provide a pH, which is compatible with the target mucous membranes such as those of the eye and any contact lenses with which the composition is intended to be used. Generally, for ophthalmic compositions a pH close to that of human tears, such as a pH of about 7.45, is very useful, although a wider pH range from about 5 to about 9, more preferably about 6.5 to about 8.5 and still more preferably about 6.5 to about 7.5 is also acceptable. These pH values and ranges generally also apply to mucomimetic compositions that are intended for administration to other mucous membranes.

The osmolarity of the present mucomimetic compositions may be adjusted with tonicity agents to a value that is compatible with the intended use of the compositions. For example, the osmolarity of ophthalmic solutions of the invention may be adjusted to approximately the osmotic pressure of normal tear fluid, which is equivalent to about 0.9 w/v % of sodium chloride in water. Examples of suitable tonicity adjusting agents include, without limitation: chloride salts of sodium and potassium; dextrose; glycerol; propylene glycol; mannitol; sorbitol; and mixtures thereof. Preferred tonicity agents are sodium chloride or combinations of sodium chloride and potassium chloride.

Tonicity agents are typically used in amounts ranging from about 0.001 to about 1% w/v. These amounts have been found to be useful in providing a physiologically acceptable tonicity. Preferably, the tonicity agent(s) will be employed in an amount to provide a final osmotic value of 150 to 450 mOsm/kg, more preferably between about 220 to about 350 mOsm/kg and most preferably between about 270 to about 310 mOsm/kg.

The compositions of the invention, particularly those that are intended for use as lens disinfection compositions, lens cleansing solutions, or lens packaging solution compositions, may be further supplemented with one or more surfactants. Such surfactants may include polyethylene oxide (PEO), polypropylene oxide (PPO), block copolymers such as polyoxamers and polyoxamines or polysorbates such as polysorbates 20 and 80. A preferred surfactant for inclusion with compositions of the invention is polysorbate 20. Inclusion of such surfactants results in effective lens cleaning during lens treatment without substantially affecting the antimicrobial activity of the compositions. The concentration of surfactants in the compositions typically ranges from about 0.01 to about 1% w/v.

The compositions of the invention may further include one or more viscosity-modifying agents such as cellulose polymers, including hydroxypropylmethyl cellulose, hydroxyethyl cellulose, ethylhydroxyethyl cellulose, hydroxypropyl cellulose, methyl cellulose, glycerol, carbomers, polyvinyl alcohol, polyvinyl pyrrolidone, carrageenans, guar, karaya, agarose, locust bean, tragacanth and xanthan gums. Such viscosity modifying components are typically employed in an amount effective to provide a desired lubricating effect to the present compositions. The concentration of such viscosity-modifying agents will typically be between about 0.01 and 0.2% w/v. Preferred viscosity modifying agents are hydroxypropylmethyl cellulose and hydroxyethyl cellulose.

A preferred composition of the present invention to be used as an ophthalmic solution comprises polyaminopropyl biguanide in an amount ranging from about 0.0001 to about 0.0005% w/v, a magnesium, calcium or magnesium/calcium complex of an anionic polymer in an amount ranging from about 0.01 to about 0.25% w/v, a viscosity-modifying agent in an amount ranging from about 0.01 to about 0.2% w/v, a surfactant in an amount ranging from about 0.01 to about 1% w/v and a tonicity agent in an amount ranging from about 0.001 to about 1% w/v. The most preferred ophthalmic solution also comprises a buffer, preferably a borate buffer. In this most preferred ophthalmic solution, the anionic polymer is hyaluronate, preferably of a molecular weight from about 70,000 to about 4 million Dalton, the viscosity-modifying agent is hydroxypropylmethyl cellulose or hydroxyethyl cellulose, the surfactant is polysorbate 20 and the tonicity agent is sodium chloride or a combination of sodium chloride and potassium chloride.

Methods for using the ophthalmic solutions described herein are considered to be within the scope of the present invention. The mucomimetic compositions of the invention may be used in wipes, solutions, lubricating products and the like that are brought in contact with other mucous membranes of a subject. They may serve to moisturize, lubricate, clean and/or disinfect. They may also be used as non-irritating, preservative or disinfecting carriers of drug substances for delivery of the drug substances into or through mucous membranes. When used as ophthalmic solutions, the compositions may also be utilized as contact lens disinfection compositions, lens cleansing solution compositions, lens packaging solution compositions or eye drops such as rewetters and tears. The compositions are highly comfortable to the eye and are biocompatible with contact lenses. The polymeric anionic and cationic compounds are known not to penetrate into the lens matrix. Unlike prior art compositions, they do not cause a discoloring of soft contact lenses, especially of silicon hydrogel lenses.

The invention is further elaborated by the following examples. The examples are provided for purposes of illustration to a person skilled in the art and are not intended to be limiting the scope of the invention as described in the claims. Thus, the invention should not be construed as being limited to the examples provided, but should be construed to encompass any and all variations that become evident as a result of the teaching provided herein.

### EXAMPLES

### Components of the Compositions of the Invention

All components of the mucomimetic compositions of the present invention can be obtained from commercial sources. Polyaminopropyl biguanide can be procured from Thor Specialties, Inc. (Trumbull, Conn., USA). All other components are available from, e.g., from Sigma-Aldrich Fluka (Buchs, Switzerland).

### Example 1

### Antimicrobial Activity of Polyaminopropyl Biguanide in the Presence of Calcium/Magnesium Salts of Anionic Polymers

Formulations: 0.1% hyaluronic acid (A) or 0.15% carboxymethylcellulose (B), or 0.05% alginate (C); 0.01% polyaminopropyl biguanide; 0.02% calcium chloride; 0.02% magnesium chloride; 0.1% sodium hydrogenborate; pH 7.2.

Parallel cultures of different microorganisms were either exposed to formulations A, B or C for 24 hours or were incubated further in their respective growth media. At the end of the challenge period, bacterial counts (per ml) were determined.

| | Challenge | Challenge | Challenge | | | |
|---|---|---|---|---|---|---|
| | formulation | formulation | formulation | No | No | No |
| Organism | A | B | C | exposure | exposure | exposure |
| B. subtitlis | <10 | <10 | <10 | 5.8 × 10⁶ | 5.8 × 10⁶ | 5.8 × 10⁶ |
| P. aeruginosa | <10 | <10 | <10 | 3.7 × 10⁶ | 3.7 × 10⁶ | 3.7 × 10⁶ |
| S. aureus | <10 | <10 | <10 | 5.4 × 10⁶ | 5.4 × 10⁶ | 5.4 × 10⁶ |
| C. albicans | <10² | <10² | <10² | 1.3 × 10⁶ | 1.3 × 10⁶ | 1.3 × 10⁶ |

### Example 2

### Formulation of a General Mucomimetic Lubricant/Wettening Ointment

An aqueous solution is prepared by mixing 0.01% hyaluronic acid sodium (MW about 1.2 million Dalton), 0.005% polyaminopropyl biguanide, 0.025% calcium chloride, 0.2% HPMC, 10% glycerol; 0.5% sodium hydrogenborate. The solution is adjusted to pH 7.2.

### Example 3

### Formulation of a Multipurpose Contact Lens Solution

An aqueous solution is prepared by mixing 0.1% hyaluronic acid sodium (MW about 1.2 million Dalton), 0.00025% polyaminopropyl biguanide, 0.02% calcium chloride, 0.02% magnesium chloride, 0.1% HPMC, 0.025% polysorbate 20, 0.3% sodium chloride, 0.2% sodium hydrogenborate. The solution is adjusted to pH 7.2.

### Example 4

### Formulation of a Dry Eye Ointment

An aqueous solution is prepared by mixing 0.2% hyaluronic acid sodium (MW about 1.2 million Dalton), 0.0003% polyaminopropyl biguanide, 0.03% calcium chloride, 0.15% HPMC, 0.2% sodium chloride, 0.3% sodium hydrogenborate. The solution is adjusted to pH 7.0.

### Example 5

### Formulation of a Glaucoma Ointment

An aqueous solution is prepared by mixing 0.25% timolol maleate ((−)-1-(tert-butylamino)-3-[(4-morpholino-1,2,5-thiadiazol-3-yl)oxy]-2-propanol maleate (1:1) (salt)), 0.25% hyaluronic acid sodium (MW about 1.2 million Dalton), 0.0002% polyaminopropyl biguanide—borate, 0.4% sodium chloride, 0.042% calcium chloride, 0.1% sodium hydrogenborate. The solution is adjusted to pH 6.5.

1. An ophthalmic solution comprising a cationic multimeric antimicrobial agent and a magnesium, calcium or magnesium/calcium complex of an anionic polymer but not myristamidopropyl dimethylamine or a nutritive base comprising a multiplicity of amino acids and vitamins, wherein the cationic multimeric antimicrobial agent is present in an amount ranging from about 0.0001 to about 0.0005% w/v and the magnesium, calcium or magnesium/calcium complex of an anionic polymer in an amount ranging from about 0.01 to about 0.25% w/v.

2. The ophthalmic solution of claim 1 wherein the multimeric antimicrobial agent is polyaminopropyl biguanide or an acceptable salt thereof.

3. The ophthalmic solution of claim 1 wherein the anionic polymer is selected from the group consisting of hyaluronate, alginate, carboxymethyl cellulose, chondroitin sulfate and mixtures thereof.

4. The ophthalmic solution of claim 1 wherein the anionic polymer has a molecular weight of from about 70,000 to about 4 million Daltons.

5. The ophthalmic solution of claim 1 further including a viscosity-modifying agent selected in an amount ranging from about 0.01 to about 0.2% w/v.

6. The ophthalmic solution of claim 5 wherein the viscosity-modifying agent is selected from the group consisting of cellulose polymers including hydroxypropylmethyl cellulose, hydroxyethyl cellulose, ethylhydroxyethyl cellulose, hydroxypropyl cellulose, methyl cellulose, glycerol, carbomers, polyvinyl alcohol, polyvinyl pyrrolidone, carrageenans, guar, karaya, agarose, locust bean, tragacanth and xanthan gums.

7. The ophthalmic solution of claim 5 wherein the viscosity-modifying agent is hydroxypropylmethyl cellulose or hydroxyethyl cellulose.

8. The ophthalmic solution of claim 1 further including a surfactant in an amount ranging from about 0.01 to about 1% w/v.

9. The ophthalmic solution of claim 8 wherein the surfactant is selected from the group consisting of polyethylene oxide, polypropylene oxide, polyoxamers, polyoxamines, polysorbate 20 and polysorbate 80.

10. The ophthalmic solution of claim 8 wherein the surfactant is polysorbate 20.

11. The ophthalmic solution of claim 1 further including a buffer in an amount ranging from about 0.01 to about 0.25% w/v.

12. The ophthalmic solution of claim 11 wherein the buffer is selected from the group of buffers consisting of borate, citrate, acetate, histidine, tris, bis-tris and mixtures thereof.

13. The ophthalmic solution of claim 1 further including a tonicity agent in an amount ranging from about 0.001 to about 1% w/v.

14. The ophthalmic solution of claim 13 wherein the tonicity agent is selected from the group consisting of sodium chloride, potassium chloride, dextrose, glycerol, propylene glycol, mannitol, sorbitol and mixtures thereof.

15. The ophthalmic solution of claim 13 wherein the tonicity agent is sodium chloride, potassium chloride or a mixture thereof.

16. An ophthalmic solution comprising polyaminopropyl biguanide in an amount ranging from about 0.0001 to about 0.0005% w/v, a magnesium, calcium or magnesium/calcium complex of an anionic polymer of a molecular weight from about 70,000 to about 4 million Dalton in an amount ranging from about 0.01 to about 0.25% w/v, a viscosity-modifying agent in an amount ranging from about 0.01 to about 0.2% w/v, a surfactant in an amount ranging from about 0.01 to about 1% w/v, a tonicity agent in an amount ranging from about 0.001 to about 1% w/v, and a buffer in an amount ranging from about 0.01 to about 0.25% w/v, whereby the pH of the aqueous solution is adjusted to be from about 6.5 to about 8.5.

17. The ophthalmic solution of claim 16 wherein the anionic polymer is hyaluronate; the viscosity-modifying agent is selected from the group consisting of cellulose polymers including hydroxypropylmethyl cellulose, hydroxyethyl cellulose, ethylhydroxyethyl cellulose, hydroxypropyl cellulose, methyl cellulose, glycerol, carbomers, polyvinyl alcohol, polyvinyl pyrrolidone, carrageenans, guar, karaya, agarose, locust bean, tragacanth and xanthan gums; the surfactant is selected from the group consisting of polyethylene oxide, polypropylene oxide, polyoxamers, polyoxamines, polysorbate 20 and polysorbate 80; the tonicity agent is selected from the group consisting of sodium chloride, potassium chloride, dextrose, glycerol, propylene glycol, mannitol, sorbitol and mixtures thereof; and the buffer is selected from the group of buffers consisting of borate, citrate, acetate, histidine, tris, bis-tris and mixtures thereof.

18. The ophthalmic solution of claim 17 wherein the viscosity-modifying agent is hydroxypropylmethyl cellulose or hydroxyethyl cellulose, the surfactant is polysorbate 20, the tonicity agent is sodium chloride or a mixture of sodium chloride and potassium chloride, and the buffer is a borate buffer.

19. An ophthalmic composition consisting of an aqueous solution of (i) polyaminopropyl biguanide in an amount ranging from about 0.0001 to about 0.0005% w/v, (ii) a magnesium, calcium or magnesium/calcium complex of an anionic polymer of a molecular weight from about 70,000 to about 4 million Dalton in an amount ranging from about 0.01 to about 0.25% w/v, (iii) a viscosity-modifying agent in an amount ranging from about 0.01 to about 0.2% w/v, (iv) a surfactant in an amount ranging from about 0.01 to about 1% w/v, (v) a tonicity agent in an amount ranging from about 0.001 to about 1% w/v, and (vi) a buffer in an amount ranging from about 0.01 to about 0.25% w/v, whereby the pH of the aqueous solution is adjusted to be from about 6.5 to about 8.5.

20. The ophthalmic composition of claim 19, wherein the anionic polymer is hyaluronate; the viscosity-modifying agent is selected from the group consisting of cellulose polymers including hydroxypropylmethyl cellulose, hydroxyethyl cellulose, ethylhydroxyethyl cellulose, hydroxypropyl cellulose, methyl cellulose, glycerol, carbomers, polyvinyl alcohol, polyvinyl pyrrolidone, carrageenans, guar, karaya, agarose, locust bean, tragacanth and xanthan gums; the surfactant is selected from the group consisting of polyethylene oxide, polypropylene oxide, polyoxamers, polyoxamines, polysorbate 20 and polysorbate 80; the tonicity agent is selected from the group consisting of sodium chloride, potassium chloride, dextrose, glycerol, propylene glycol, mannitol, sorbitol and mixtures thereof; and the buffer is selected from the group of buffers consisting of borate, citrate, acetate, histidine, tris, bis-tris and mixtures thereof.

21. The ophthalmic composition of claim 20, wherein the viscosity-modifying agent is hydroxypropylmethyl cellulose or hydroxyethyl cellulose, the surfactant is polysorbate 20, the tonicity agent is sodium chloride or a mixture of sodium chloride and potassium chloride, and the buffer is a borate buffer.

22. A composition according to any of the above claims wherein the cationic multimeric antimicrobial agent is present in an amount ranging from about 0.001 to about 0.01% w/v.
